(19) United States
(12) Patent Application Publication
(10) Pub. No.: US 2010/0049286 A1
(43) Pub. Date: Feb. 25, 2010

(54) METHOD AND SYSTEM FOR KILING LICE AND NITS

(76) Inventor: Roland Thorsen, Abyhoj (DK)

Correspondence Address: JAMES C. WRAY, 1493 CHAIN BRIDGE ROAD, SUITE 300, MCLEAN, VA, 22101, US

(21) Appl. No.: 12/448,185

(22) PCT Filed: Dec. 12, 2007
(86) PCT No.: PCT/DK2007/000543

§ 371 Date: Sep. 15, 2009

(30) Foreign Application Priority Data
Dec. 12, 2006 (DK) PA 200601628

(51) Int. Cl. A61F 7/00

(52) U.S. Cl. 607/107

(57) ABSTRACT

The system includes means for providing different temperature, in the inner air injection area and the outer air injection area. Due to the temperature impact the lice will be stimulated to leave the scalp and be guided into or through inner membrane, where an additional temperature action will kill the lice.

### FIELD OF THE INVENTION

The present invention concerns a system and a method for treating an area with lice and/or nits, mainly lice and nits present on the scalp and in the hair of the head on a human, the system including
largely airtight enclosing means for enclosing an area with lice and/or nits;
heating means for injecting and heating air, the heating means including means for controlling the temperature of the injected air and means for regulating the duration of the air injection.

### BACKGROUND OF THE INVENTION

Lice belongs to a group of insects living on animals and humans. The head lice occurring on the scalp and in the hair of humans are particularly adapted to structure which is peculiar for the hair appearing on the heads of humans. The structure of the head hair differs e.g. from beards and hair under the arms. Lice may thus climb in the hair appearing on the heads of humans, but not in beards and hair under the arms.

Nits die at about 48° C., while lice die by heating to about 52° C. for about 10-11 minutes. Lice may, however, make themselves heat-resistant by a hormonal process. The heat resistance is achieved in that the lice excreting a heat resistant and protecting secrete through their outer skeleton. This is part of the natural defence mechanism of the lice. When the lice have become heat-resistant, they may tolerate very high temperatures (above 100° C.). The presence of a humid environment improves the lice's ability of bringing themselves into a heat-resistant condition. In connection with heat treatment with the intention of exterminating lice, it is thus important that the hair is dry. It is important to consider the defence mechanisms of the lice in order to have success with methods for exterminating lice based on heat treatment.

Through the years, several methods for treating lice and nits have been presented. EP 0 221 004 describes a plastic or rubber hood in which the hair is enclosed. By substituting oxygen with a gas it is possible to suffocate lice and eggs. This form of treatment is connected with a number of drawbacks. In order for the gas not to escape the hood, the hood has to fit completely tight, which is problematic. Furthermore, it is expensive and impractical to use gas which necessarily has to be store in a container, a fact making it unsuited for common application in a home.

U.S. Pat. Nos. 5,887,357 and 5,887,357 describe hoods which may be used for treating lice and nits. None of these hoods provide efficient treatments, as they do not take into consideration that the lice rapidly may adapt and hereby protect themselves against the impact of very high temperatures.

WO 2005102036 discloses a method and a hood for treating lice and nits. The hair is inserted in an airtight hood in which is blown hot air. Initially, 80-200° C. hot air is blown into the hood. Subsequently, air at 45-70° C. is blown into the hood for a longer period of time. It is a drawback that the lice, whether dead or alive, remain in the hair since a large part of the lice, due to the strong heat impact, get to make themselves resistant to the heat impact in time and are thus not killed in connection with the heating process.

### OBJECT OF THE INVENTION

The purpose of the present invention is to provide a system and a method for treating lice and nits, where the lice are stimulated to leave the hair and be guided into an area where an action of temperature will kill the lice, so that one may do away with the lice that are not dead afterwards, by emptying or destructing the hood. It is furthermore a purpose of the invention to provide a system and a method for treating lice and nits, which is user-friendly, harmless for the user, applicable to all and which provides an efficient and trustworthy removal of the lice from the hair.

### DESCRIPTION OF THE INVENTION

According to the present invention this is achieved by a system for treating an area with lice and/or nits, mainly lice and nits present on the scalp and in the hair of the head on a human, the system including:
largely airtight enclosing means for enclosing an area with lice and/or nits;
heating means for injecting and heating air, the heating means including means for controlling the temperature of the injected air and means for regulating the duration of the air injection;
   which is peculiar in that one or more areas with great air permeability are provided in the enclosing means for enclosing an area with lice and/or nits. Areas with great air permeability may e.g. be openings, holes, perforations, valves and similar.

The method according to the invention consists of
enclosing an area with lice and/or nits with enclosing means;
heating the area with lice and/or nits by injecting air heated by using heating means; and
removing the applied enclosing means from the area with lice and/or nits;
controlling the injected air via one or more areas with great air permeability provided in the enclosing means.

By the system and the method according to the invention is hereby achieved possibility of providing control of the airflow through the applied enclosing means. By using areas with great air permeability in the shape of e.g. holes, the air flow may advantageously be controlled according to actual needs and wishes. The airflow is controlled in that, due to the injecting of air into the enclosing means, an overpressure arises, and the air will blow out through the openings as a lower pressure is present here. Hereby it is possible to provide an optimal airflow for heating in connection with treating an area with lice and/or nits. Hereby it becomes possible to conduct air currents to central areas, including areas around and behind the ears.

According to a preferred embodiment, the system according to the invention is peculiar in that the enclosing means for enclosing an area with lice and/or nits include an inner air injection area provided between the head and an inner membrane and an outer air injection area provided between an inner membrane and an outer membrane.

Hereby is achieved the possibility of varying the temperature in the inner air injection area and the outer air injection area, respectively. Hereby is achieved possibility of providing thermal conditions that stimulate the lice to leave the hair and the scalp and be attached to or penetrate through the inner membrane. After that, it is possible to inject air in the outer air injection area and/or the inner membrane at such a high temperature that the lice die or are made incapable of leaving the inner membrane. Hereby it is avoided that lice in connection with establishing heat resistance remain in the hair in living condition after the treatment.

According to a preferred embodiment, the system according to the invention is peculiar in that it includes means for providing different temperatures in the inner air injection area and the outer air injection area, respectively. Hereby is achieved the possibility of varying and maintaining different temperatures in the inner air injection area and the outer air injection area, respectively. This enables a number of treatment methods, including the possibility of providing a lower temperature in the inner injection area than in the outer injection area. The means for providing different temperatures hereby ensure that the lice due to the temperature impact will be stimulated to leave the scalp and the hair and be guided into or through the inner membrane where an additional temperature action with kill the lice. Thus it will be possible to get rid of the lice which are not killed by emptying the enclosure means. It is possible to use an inner membrane which the lice can move through.

According to yet a preferred embodiment, the system according to the invention is peculiar by including an inner membrane with a structure that enable the lice to be attached to the inner membrane or to move through the inner membrane. Hereby is ensured that lice which (due to established heat resistance) do not die in connection with the heat action may be removed together with the enclosing means. It is possible to use a structure that is similar to the structure of head hairs, which, simply said, are structured like bamboo plants with serially joined sections. It is the joints of the sections that the lice utilise when they move in head hair.

An example of an inner membrane to which the lice may attach is a membrane made in so-called 3D knitting technology or 3D weaving technology. Membranes made by these methods are distinguished by having two outer fabric layers separated by an open intermediate layer. The intermediate layer typically consists of so-called Z-binder yarn connecting the two outer layers in a random structure, and where the yarns may be selected from various raw materials, as e.g. glass, polymers like polypropylene, polyethylene and the like, or carbon, whereby it is possible to form the intermediate layer with very complex structures, so that when the lice have penetrated through the first layer, they will be able to attach to the intermediate layer so that when the enclosing means are removed from the head, thereby removing the inner membrane, the lice caught in the inner membrane will thus be removed together with the enclosing means. Another suitable material is a so-called 3D knitted sandwich structure where two individual textiles are tied together with a structure where several needles penetrate both textiles in different directions with different materials, thereby forming a zone between the two textiles consisting of relatively regular structure with many cavities and in particular many fibres of various types. In technical language, these structures are known as sandwich or spacer structures.

Another example of a material that may be used for the inner membrane is a polymer material in which is incorporated so-called conductive polymer valves, which are polymer valves that are actuated because of changes in temperature. By designing the polymer to be active at e.g. 43° C., it may be achieved that the lice get through the valve openings before the valves close, so that the lice will be moved from the hair and the scalp and out into the area outside the inner membrane. A similar technology, where a bimetallic film is used in connection with the inner membrane, may also produce through-flow of air, however also of lice, until a certain temperature is reached, after which the bimetal, due to the properties of the bimetal known from e.g. bimetallic switches (flashlight) may be acted on to close and thereby switch off the airflow and the passage of lice as well. In the way described above it will be possible to get the lice to travel from the hair and scalp through the membrane and out into the area outside the inner membrane, and by closing the bimetallic valve prevent the lice from going back into the hair. Then the enclosing means may be destroyed or the lice caught between the inner membrane and the outer membrane may be killed, e.g. by injecting very hot air. Injecting very hot air in this cavity will not cause inconvenience to a user, as the air space between the scalp and the inner membrane will provide insulation regarding this heating.

According to a preferred embodiment, the method according to the invention is peculiar in that the temperature in the inner air injection area and the temperature in the outer air injection area, respectively, are regulated by heating means disposed outside the enclosing means. Such a method is very advantageous as very limited demands are made to the nature of the enclosing means. Thus there is no need for placing sensors in the enclosing means. It is thus possible to provide a single system where the means for regulating the temperature in the inner air injection area and the outer air injection area are regulated by heating means disposed outside the enclosing means.

It is possible to perform treatment of an area with lice and/or nits by initially injecting heated air into the inner injection area for stimulating the lice to attach to the inner membrane. It is also possible initially to inject air into the inner injection area as well as the outer injection area. If the latter method is applied, it is to be ensured that the lice are stimulated to move, which may be effected by providing a temperature attractive to the lice in the outer injection area and a less attractive temperature (e.g. a high temperature) in the inner injection area.

It is advantageous to use valve holes in the outer membrane for controlling the flow of the air. For example, 6-10 holes may be provided in the outer membrane.

It may be advantageous to provide the injection of air from the heating means to the enclosing means in such a way that the air is blown in close to the back of the head and then moved up and out along the head. The air will hereby mainly be moved against the hairs. This ensures that the air is moved right into the scalp so that lice and nits, which are also situated there, are also influenced by temperature.

### DESCRIPTION OF THE DRAWING

In the following, the invention will be explained in more detail with reference to the enclosed drawing where:

FIG. 1 shows a view of a human provided with a hood which is connected to a blower;

FIG. 2 shows a close-up view of a detail of a head clothed with a hood;

FIG. 3 shows a view of a sectioned blower; and

FIG. 4 shows a human provided with a sectioned hood.

### DETAILED DESCRIPTION OF THE INVENTION

Firstly, it is to be noted that the attached illustration only illustrates a non-limiting embodiment of the invention. Other embodiments will be possible within the scope of the present invention as defined in the claims.

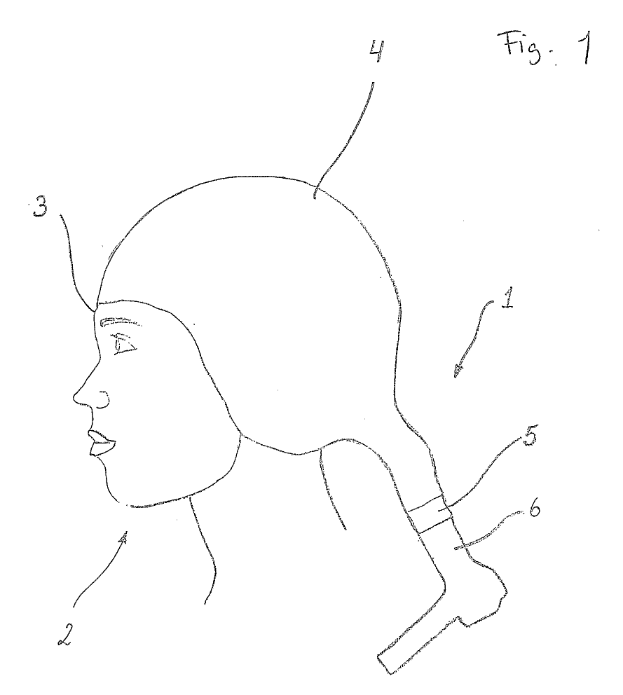

FIG. 1 shows an embodiment of a system 1 according to the invention. A person 2 is dressed with a hood 4 which via an adapter 5 is connected to a blower 6. During use, the hood is put on so that it fits tightly to the person's head 3. Hereby is ensured that the injected air is held within the enclosed area.

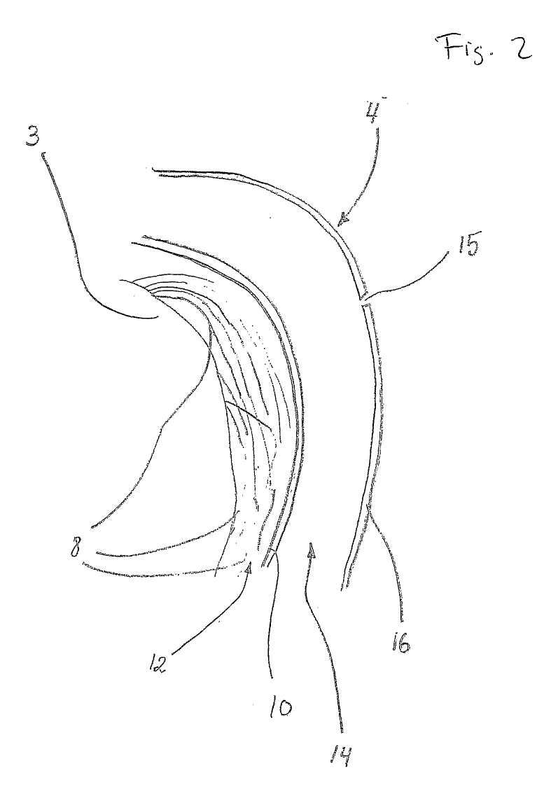

FIG. 2 shows a close-up view of a detail of a head 3 dressed with a hood 4. The head 3 includes a number of hairs 8. The hood 4 includes an inner membrane 10 and an outer membrane 16 in which is provided an aperture 15. Between the head 3 and the inner membrane 10 is provided an inner air injection area 12, whereas between the inner membrane 10 and the outer membrane 16 is provided an outer air injection area 14. During use, air may optionally be injected into the inner air injection area 12 and/or the outer injection area 14. Air injected into the outer injection area 14 may leave the outer injection area through the aperture 15. Advantageously, more openings may be provided, e.g. in a number of 6-10.

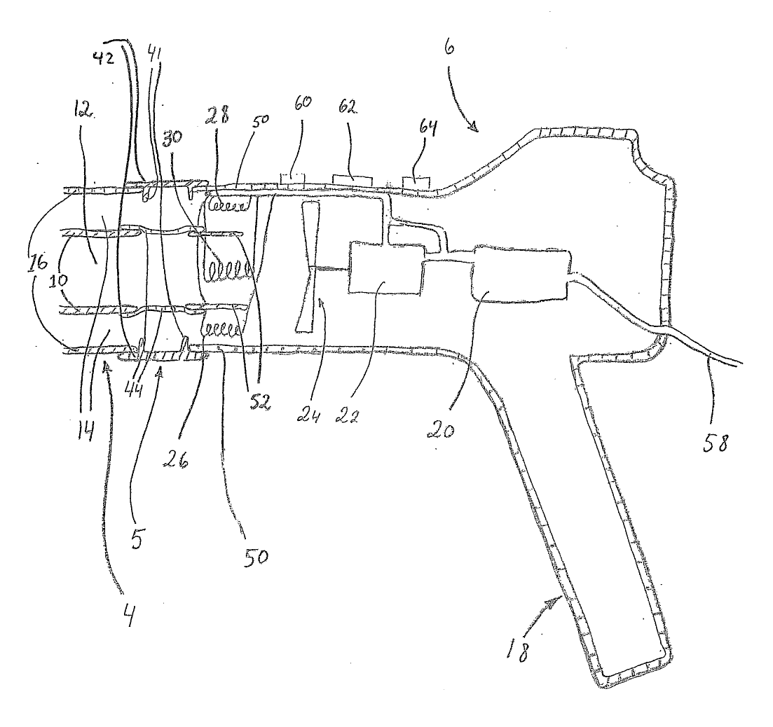

FIG. 3 shows a longitudinal section of an embodiment of a blower 6 for use in connection with the invention. The blower 6 is connected to an adapter 5 and to a hood 4. The blower 6 includes a largely pistol-shaped housing 50 with adjacent handle 18. Within the house 50 is provided an electric motor 22 which is connected to a propeller 24. Moreover, a printed circuit board 20 operating as control unit is provided in the housing 50. The control unit is powered by electricity through a line 58. The control unit 20 is connected to the electric motor 22 and three filaments 26, 28, 30 provided in the housing 50. These filaments are disposed in channels separated by the wall of the housing 50 and separating walls 52, respectively. The power supply to the three filaments 26, 28, 30 is controlled by the control unit 20. At the outer side of the housing 50, an indicator device 60, a regulating device 62 and a switch 64 are provided, all connected to the control unit 20. The electric control of blowers 6 (including the parts it includes) may be provided by the skilled in the art.

The blower 6 is connected to an adapter 5. The adapter S comprises plate-shaped outer sides 42 on which are provided stop flanges 41 that limit the extent to which the blower 6 and the outer membrane 16 of the hood may be moved into the adapter 5. At one side, the adapter 5 is connected to the housing 50, and at the other side, it is connected to the outer membrane 16 of the hood. The adapter 5 includes flexible partitionings 44 which via deformation provided in connection with mounting of blower 6 and hood 4, respectively, apply a force against the partitionings 52 of the housing and the inner membrane 10 of the hood. Hereby, the adapter 5 is secured in engagement with the blower 6 and the hood 4.

During use, the switch 64 may be used for switching the blower 6 on and off, and in addition, the regulating device 62 may be used for regulating the temperature of the air blown into the inner injection area 12 and the outer injection area 14, respectively. The regulating device 62 may furthermore be used for determining the duration of the injection and the flow rate of the injected air. The indicator 60 is equipped with a diode which e.g. may be used for signalling that the treatment is finished. The indicator 60 furthermore includes a sound unit in the form of a loudspeaker that may emit a sound signal, e.g. in connection with start and finish of the treatment. The indicator may furthermore contain a display showing remaining treatment time.

The regulating device 62 may advantageously include a number of preset treatment programs. Among these preset treatment programs there may advantageously be programs where for a short time period (e.g. for 3 s), very hot air (e.g. 180° C.) may initially be blown into ducts leading to the inner air injection area 12 and/or into the outer air injection area 14 to heat tubes and membranes. The deposited energy thus goes to increasing the temperature of tubes and membranes. Subsequently, about 40° C. hot air is injected into the inner air injection area 12 and/or into the outer air injection area 14 for 2-10 minutes. An air temperature of 40° C. will stimulate the lice to move away from the hair and up into the inner membrane 10. Among the preset treatment programs there may also advantageously be programs where initially for a short time period (e.g. for 3 s) very hot air (e.g. 180° C.) is blown into ducts leading to the inner air injection area 12 and/or into ducts leading to the outer air injection area 14, after which about 60° C. hot air is injected into the inner air injection area 12 and/or into the outer air injection area 14 for 10-11 minutes. Hereby is ensured that nits and lice that have not become heat resistant die.

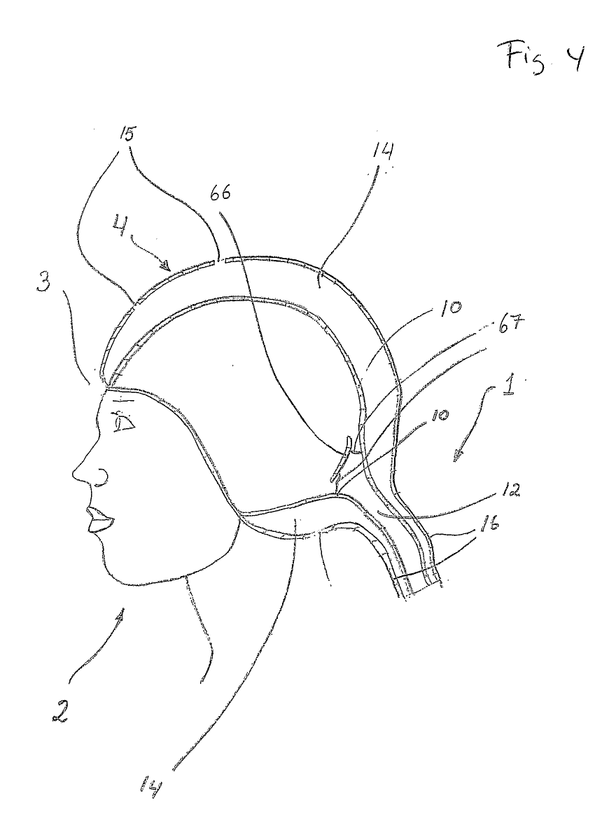

FIG. 4 shows a person 2 dressed in a sectioned hood 2 on the head 3. The hood 4 includes an inner membrane 10, an outer membrane 16 and a heat shield 66 connected to the inner membrane 10 by cords 67. The heat shield 66 protects the user against strong heat action in connection with injection of hot air into the inner air injection area 12. Holes 15 are provided in the outer membrane 16. The inner membrane 10 may advantageously be adhesive. Hereby, the inner membrane 10 may better fit tightly to the head 3.

It is possible to use a hood that only includes a single membrane 10, corresponding to the inner membrane 10 shown in FIG. 4. Thus it will still be possible to perform a treatment where the lice are stimulated to crawl in the inner membrane 10, as a consequence of injecting hot air.

1. A system (1) for treating an area with lice and/or nits, mainly lice and nits present on the scalp and in the hair (8) of the head (3) on a human (2), the system including
largely airtight enclosing means (4) for enclosing an area with lice and/or nits;
heating means (6) for injecting and heating air, the heating means (6) including means for controlling the temperature of the injected air and means for regulating the duration of the air injection;
wherein one or more areas with great air permeability (15) are provided in the enclosing means (4) for enclosing an area with lice and/or nits, and that the enclosing area includes an inner air injection area (12) provided between the head (3) and an inner membrane (10) and an outer air injection area (14) provided between an inner membrane (10) and an outer membrane (16).

2. System according to claim 1, wherein the system (1) includes means for providing different temperatures in the inner air injection area (12) and the outer air injection area (14), respectively.

3. System according to claim 1, wherein an inner membrane (10) with a structure enabling lice to be attached in the inner membrane (10).

4. A method for treating an area with lice and/or nits, mainly lice and nits present on the scalp and in the hair (8) of the head (3) on a human (2), the method consisting of:
enclosing an area with lice and/or nits with enclosing means (4);
heating the area with lice and/or nits by injecting air streams heated by using heating means (6); and
removing the applied enclosing means (4) from the area with lice and/or nits;
where the injected air streams are controlled via one or more areas with great air permeability (15) provided in the enclosing means (4).

5. Method according to claim 4, wherein by injecting air, different temperatures are provided in
an inner air injection area (12) located between the area with lice and/or nits and the part of the enclosure means (4) which during use of the enclosing means (4) borders on the area with lice and/or nits, and
an outer air injection area (14), the outer air injection area (14) bordering on the inner air injection area (12), respectively.

6. Method according to claim 4, wherein the temperature in the inner air injection area (12) and the temperature in the outer air injection area (14), respectively, are regulated by heating means (6) disposed outside the enclosing means (4).

7. Method according to claim 4, wherein heating of the area with lice and/or nits is initially effected by injecting air streams heated to 100-250° C. for a time period of 0.5-10 seconds and subsequently by injecting air streams heated to 40-70° C. for a time period of 1-20 minutes by using heating means (6).

8. Method according to claim 4, wherein the heating of the area with lice and/or nits is effected by injecting air streams heated to 40-70° C. for a time period of 1-20 minutes.
